(19) United States
(12) Patent Application Publication
(10) Pub. No.: US 2020/0089713 A1
(43) Pub. Date: Mar. 19, 2020

(54) SYSTEM AND METHOD FOR CRAWLING

(71) Applicant: Innoplexus AG, Eschbom (DE)

(72) Inventors: Gaurav Tripathi, Pune (IN); Vatsal Agarwal, Rampur (IN); Govardhan Veer, Ahmednagar (IN)

(21) Appl. No.: 16/366,544

(22) Filed: Mar. 27, 2019

(30) Foreign Application Priority Data
Mar. 27, 2018 (GB) 1804920.5

(51) Int. Cl. G06F 16/951; G06F 16/955; G16H 50/70

(52) U.S. Cl. CPC G06F 16/951; G16H 50/70; G06F 16/955

(57) ABSTRACT

A system and method of crawling. Furthermore, the system includes a data processing arrangement including a communication interface for accessing a wide area computer network and a crawling module. Furthermore, the crawling module is operable to receive a Uniform Resource Identifier; retrieve source information associated with the Uniform Resource Identifier, wherein the source information includes a pool of data elements; determine a relevant data element from the pool; analyze the relevant data element to determine an importance factor associated therewith; assign a chronological score to the relevant data element based on the importance factor; and crawl the relevant data element based on the assigned chronological score. Additionally, a database arrangement is communicably coupled to the data processing arrangement, operable to aggregate the at least one relevant data element based on the assigned chronological score.

### CROSS-REFERENCE TO RELATED APPLICATIONS

This application claims the benefit under 35 U.S.C. § 119(a) and 37 CFR § 1.55 to UK Patent Application No. GB1804920.5, filed on Mar. 27, 2018, the entire content of which is incorporated herein by reference.

### TECHNICAL FIELD

The present disclosure relates generally to computer networks; and more specifically, to systems that crawl. Furthermore, the present disclosure relates to methods of (for) crawling. Moreover, the present disclosure also relates to computer readable medium containing program instructions for execution on a computer system, which when executed by a computer, cause the computer to perform method steps of crawling.

### BACKGROUND

In recent years, there has been an explosion of information on the World Wide Web (www). Essentially, the information is available on the World Wide Web in a form of web pages. Additionally, the web pages are electronically stored in their respective websites on a server. Furthermore, with the creation of millions of web pages, web crawlers or web spiders are conventionally employed for the extraction of useful information from the websites identified by Uniform Resource Identifiers (URI). Additionally, the web crawlers use the Uniform Resource Identifiers associated with the servers to download and upload information. Thus, the aforesaid web crawlers function as “robotic devices” that crawl around web pages and interrogate them for their information.

However, conventional processes of crawling web pages encounter several problems. In earlier days, the web crawlers were able to perform crawling processes more efficiently, owing to a lesser number of websites and a relatively static nature of the websites. However, the more recently designed websites have evolved to become more dynamic. Typically, the dynamic websites obstruct the aforesaid process of crawling. Additionally, the process of crawling is interrupted by leading the web crawler to dummy websites. Furthermore, there are contemporarily employed crawling operations that are also interrupted by pushing a given web crawler in an infinite loop of Uniform Resource Identifiers.

Existing crawling systems employ cookies, Application Programming Interface (API), breaking of Captcha and so forth to crawl such dynamic websites. However, the aforesaid procedures are performed manually to overcome the obstructions faced during crawling. Furthermore, the aforementioned procedures are unreliable for identifying the dummy websites or the infinite loops of Uniform Resource Identifiers efficiently.

Therefore, in light of the foregoing discussion, there exists a need to overcome the aforementioned drawbacks associated with the conventional methods of (for) crawling the websites, and also associated with systems that employ aforesaid methods for performing crawling activities.

### SUMMARY

The present disclosure seeks to provide a system that crawls. The present disclosure also seeks to provide a method of (for) crawling. The present disclosure also seeks to provide a computer readable medium, containing program instructions for execution on a computer system, which when executed by a computer, causes the computer to perform method steps for crawling. The present disclosure seeks to provide an at least partial solution to the existing problem of tedious and manual methods of web crawling. An aim of the present disclosure is to provide a solution that overcomes at least partially the problems encountered in prior art, and provides a faster and efficient system for web crawling. Moreover, the present disclosure provides an optimal system for substantially reducing manual intervention required in crawling.

In one aspect, an embodiment of the present disclosure provides a system that crawls, wherein the system comprises:
a data processing arrangement comprising a communication interface for accessing a wide area computer network and a crawling module, wherein the crawling module is operable to:
   receive at least one Uniform Resource Identifier;
   retrieve source information associated with the at least one Uniform Resource Identifier, wherein the source information includes a pool of data elements;
   determining at least one relevant data element from the pool of data elements, wherein determining the at least one relevant data element includes:
      identifying at least one attribute associated with each data element in the pool of the data elements,
      analyzing the at least one identified attribute, based on predefined qualifier conditions, for detecting a relevance factor for the each data element, and
      using the relevance factor to determine the at least one relevant data element from the pool of data elements;
   analyze the at least one relevant data element to determine an importance factor associated therewith;
   assign a chronological score to each of the at least one relevant data element based on the determined importance factor thereof; and
   crawl each of the at least one relevant data element based on the assigned chronological score thereof; and
a database arrangement communicably coupled to the data processing arrangement, wherein the database arrangement is operable to aggregate the at least one relevant data element based on the assigned chronological score.

In another aspect, an embodiment of the present disclosure provides a method that crawls, wherein the method includes using a computer system, wherein the method comprises:
(i) receiving at least one Uniform Resource Identifier;
(ii) retrieving source information associated with the at least one Uniform Resource Identifier, wherein the source information includes a pool of data elements;
(iii) determining at least one relevant data element from the pool of data elements, wherein determining the at least one relevant data element includes:
   identifying at least one attribute associated with each data element in the pool of the data elements,
   analyzing the at least one identified attribute, based on predefined qualifier conditions, for detecting a relevance factor for the each data element, and
   using the relevance factor to determine the at least one relevant data element from the pool of data elements;
(iv) analyzing the at least one relevant data element to determine an importance factor associated therewith; and
(v) assigning a chronological score to each of the at least one relevant data element based on the determined importance factor thereof; and
(vi) crawling the each of the at least one relevant data element based on the assigned chronological score thereof.

In yet another aspect, an embodiment of the present disclosure provides a computer readable medium, containing program instructions for execution on a computer system, which when executed by a computer, cause the computer to perform method steps of (for) a method of crawling, the method comprising the steps of:
receiving at least one Uniform Resource Identifier;
retrieving source information associated with the at least one Uniform Resource Identifier, wherein the source information includes a pool of data elements;
determining at least one relevant data element from the pool of data elements, wherein determining the at least one relevant data element includes:
   identifying at least one attribute associated with each data element in the pool of the data elements,
   analyzing the at least one identified attribute, based on predefined qualifier conditions, for detecting a relevance factor for the each data element, and
   using the relevance factor to determine the at least one relevant data element from the pool of data elements;
analyzing the at least one relevant data element to determine an importance factor associated therewith;
assigning a chronological score to each of the at least one relevant data element based on the determined importance factor thereof; and
crawling each of the at least one relevant data element based on the assigned chronological score thereof.

Embodiments of the present disclosure substantially eliminate or at least partially address the aforementioned problems in the prior art, and enables optimized crawling of dynamic websites with substantially reduced human intervention.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is an illustration of a block diagram of a system that crawls, in accordance with an embodiment of the present disclosure;

FIG. 2 is an illustration of steps of a method of (for) crawling, in accordance with an embodiment of the present disclosure; and

FIG. 3 is an illustration of steps of a method to determine the at least one relevant data element, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

In overview, embodiments of the present disclosure are concerned with methods of (for) crawling websites, for example for crawling restricted websites, and specifically to, analysing source information associated with the websites to determine a crawling protocol thereof. The embodiments are concerned with an improved technical manner of operating data communication networks hosting websites, wherein more efficient crawling is enabled that can reduce an amount of data communicated within the data communication networks, and thereby potentially reduce energy dissipation in the data communication networks and improve their temporal responsiveness when in operation.

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In one aspect, the present disclosure provides a system that crawls, wherein the system comprises:
a data processing arrangement comprising a communication interface for accessing a wide area computer network and a crawling module, wherein the crawling module is operable to:
   receive at least one Uniform Resource Identifier;
   retrieve source information associated with the at least one Uniform Resource Identifier, wherein the source information includes a pool of data elements;
   determining at least one relevant data element from the pool of data elements, wherein determining the at least one relevant data element includes:
      identifying at least one attribute associated with each data element in the pool of the data elements,
      analyzing the at least one identified attribute, based on predefined qualifier conditions, for detecting a relevance factor for the each data element, and
      using the relevance factor to determine the at least one relevant data element from the pool of data elements;
   analyze the at least one relevant data element to determine an importance factor associated therewith;
   assign a chronological score to each of the at least one relevant data element based on the determined importance factor thereof;
   crawl each of the at least one relevant data element based on the assigned chronological score thereof; and
   a database arrangement communicably coupled to the data processing arrangement, wherein the database arrangement is operable to aggregate the at least one relevant data element based on the assigned chronological score.

In another aspect, the present disclosure provides a method that crawls, wherein the method includes using a computer system, wherein the method comprises:
(i) receiving at least one Uniform Resource Identifier;
(ii) retrieving source information associated with the at least one Uniform Resource Identifier, wherein the source information includes a pool of data elements;
(iii) determining at least one relevant data element from the pool of data elements, wherein determining the at least one relevant data element includes:
   identifying at least one attribute associated with each data element in the pool of the data elements,
   analyzing the at least one identified attribute, based on predefined qualifier conditions, for detecting a relevance factor for the each data element, and
   using the relevance factor to determine the at least one relevant data element from the pool of data elements;
(iv) analyzing the at least one relevant data element to determine an importance factor associated therewith;
(v) assigning a chronological score to each of the at least one relevant data element based on the determined importance factor thereof; and
(vi) crawling each of the at least one relevant data element based on the assigned chronological score thereof.

The present disclosure provides the aforementioned system and method of (for) crawling of websites. The described system constitutes a crawling module which is operable to retrieve automatically a source information associated with a Uniform Resource Identifier. Beneficially, the source information associated with the Uniform Resource Identifier enables the system to identify dynamic websites and dummy websites. Furthermore, the present disclosure provides a system to crawl such dynamic websites and dummy websites easily. Additionally, the present disclosure also seeks to provide a system that automatically terminates an infinite loop of Uniform Resource Identifiers. Beneficially, the present disclosure reduces human intervention in the process of crawling and further optimizes the process by improving the speed of crawling and producing relevant data.

According to the present invention, a system that crawls relates to an arrangement of modules and/or units that include programmable and/or non-programmable components; for example, the components include digital hardware, for example customer-design ASIC's and FPGA's. The programmable and/or non-programmable components are configured to identify, extract, process and provide data that enables crawling of digital content, namely web content. Throughout the present disclosure, the term “crawling” as used herein relates to the process of browsing through a network of computing devices, for example the Internet®, in a methodical and/or automated manner using a link. Furthermore, crawling includes extracting data stored in one of the computing devices of the network. Moreover, crawling refers to analyzing and indexing the extracted data in a manner that enables optimizing the process of extracting data stored in the computing devices of the network. Additionally, crawling can include one or more specifications of what to crawl, including how, when, and other parameters for controlling the process of crawling. Optionally, crawling includes extracting back data related to static data or resource files that are associated with the links. Furthermore, crawling can include extracting dynamic data from the link, such as the data downloaded from the Internet or displayed by the link, upon execution.

According to the present invention, the system comprises a data processing arrangement. Throughout the present disclosure, the term “data processing arrangement” as used herein relates to at least one programmable or computational entity configured to acquire process and/or respond to instructions for crawling. For example, the computational entity may include a memory, a network adapter and the likes. In another example, data processing arrangement includes, but are not limited to, a microprocessor, a microcontroller, a complex instruction set computing (CISC) microprocessor, a reduced instruction set (RISC) microprocessor, a very long instruction word (VLIW) microprocessor, or any other type of processing circuit for executing the instructions of crawling. Furthermore, the data processing arrangement includes one or more individual processors, processing devices and various elements of a computer system associated with a processing device that may be shared by other processing devices. Additionally, one or more individual processors, processing devices, and elements are arranged in various architectures for responding to and processing the instructions that drive the system for retrieving information, for example, resource files related to the link.

Moreover, the data processing arrangement is configured to host computer programs and/or routines that provide various services. For example, the services may include providing connectivity between the modules of the system (described hereinafter), generating an interface to enable providing input to the system, processing the extracted data generated from crawling the link, training an algorithm based on the extracted data from crawling and the likes.

The data processing arrangement comprises the communication interface for accessing the wide area computer network. Throughout the present disclosure, the term “communication interface” as used herein relates to an arrangement of interconnected components that are configured to facilitate data communication between one or more electronic devices, software modules and/or databases, whether available or known at the time of filing or as later developed. Furthermore, the communication interface facilitates data communication via a collection of interconnected (public and/or private) networks that are linked together by a set of standard protocols. Examples of standard protocols may include, but not limited to, Internet® Protocol (IP), Wireless Access Protocol (WAP), Frame Relay, Asynchronous Transfer Mode (ATM), Hypertext Transfer Protocol (HTTP), File Transfer Protocol (FTP), and the likes. Furthermore, any other suitable protocols using voice, video, data, or combinations thereof, can also be employed. The system for crawling uses the communication interface to access the wide area computer network.

Throughout the present disclosure, the term “wide area computer network” as used herein relates to a structure and/or module including interconnected computing components storing user-viewable hypertext documents (commonly referred to as Web documents or Web pages). Furthermore, the interconnected computing components form a distributed computing environment storing a distributed collection of interlinked, user-viewable hypertext documents accessible via the communication interface. Optionally, the wide area computer network can be implemented as client server architecture including client and server software components which provide access to such documents using standardized protocols. For example, standard protocol for locating and acquiring Web documents may be Hypertext Transfer Protocol (HTTP) and the Web pages are encoded using Hypertext Mark-up Language (HTML). Optionally, the wide area computer network refers to a global network of computers encompassing future mark-up languages and transport protocols that can be used in place of (or in addition to) Hypertext Mark-up Language (HTML) and Hypertext Transfer Protocol (HTTP) for communication.

The communication interface is configured to operate as an interface for the data processing arrangement to establish data communication with the wide area computer network. The data communication enables the data processing arrangement to crawl user-viewable hypertext documents. Specifically, the data communication provides an arrangement, namely a means, for the data processing arrangement to extract the user-viewable hypertext documents and associated information therein, from the computing components of the wide area computer. Examples of associated information may include static data or resource files of the user-viewable hypertext documents. Furthermore, data processing arrangement uses links to the user-viewable hypertext documents, namely Uniform Resource Locator (URL) to extract the user-viewable hypertext documents and associated information.

The data processing arrangement comprises crawling module. Throughout the present disclosure, the term “crawling module” as used herein relates to a computational unit that is operable to respond and process the instructions for carrying out web crawling. The computational unit includes hardware configured to host logic and/or collection of software instructions for performing the crawling operation. Optionally, the logic and/or collection of software instructions may include entry and exit points. Moreover, the logic and/or collection of software instructions may be written in a programming language, such as, for example, PHP®, Java®, C®, C++®, and the likes. Furthermore, the logic and/or collection of software instructions may be compiled and linked into an executable program. Optionally, the executable program is configured to perform a specific task, and more preferably refers to a computer program that is configured to automate a computing task that would otherwise be performed manually, namely crawling. Examples of the computing task may include using Uniform Resource Locator to access user-viewable hypertext documents stored in the computing components of the wide area computer network, and extracting and analyzing the user-viewable hypertext documents and static data or resource files associated to the user-viewable hypertext documents. Optionally, the executable program is a bot (or spider) that is configured to autonomously browse the wide area computer network (such as the web) to extract user-viewable hypertext documents. In such an example, the bot and/or spider may be hosted on a computing device (such as a computer, a laptop, a smartphone and the like).

Furthermore, the crawling module can be implemented using one or more individual processors, processing devices and various units associated with a processing device that may be shared by other processing devices. Additionally, the one or more individual processors, processing devices and units are arranged in various architectures for responding to and processing the instructions that drive the web crawling module to perform the web crawling. Optionally, the crawling module is implemented in a distributed architecture. Specifically, in the distributed architecture, the programs (such as the bots and/or spiders) configured to browse the wide area computer network, namely the web, are hosted on one or more computing hardware that is spatially separated from each other.

The crawling module is operable to receive at least one Uniform Resource Identifier. Throughout the present disclosure, the term “Uniform Resource Identifiers” (referred to, herein later as “URIs”) as used herein relates to any electronic object and/or link that enable locating and extracting a resource (such as the user-viewable hypertext document) stored in the computing components of the wide area computer network. For example, the URIs acts as references to web pages on the wide area computer network, namely the Internet®. In an example, the URI is a Uniform Resource Locator (referred to, herein later as “URL”). Therefore, although the exemplary embodiments are described hereinafter with respect to URLs, a scope of the claimed subject-matter is not so limited, and one or more of the described examples may be utilized in connection with the URI. In another example, the URI may include a uniform resource name (URN) and a URL. Optionally, the URI may be provided as a hyperlink. The term “hyperlink” relates to a reference that points to a resource available via a communication network and, when selected by a bot (such as computer program for web crawling), automatically navigates an application to the resource. In this regard, the hyperlink can include hypertext.

Optionally, the data processing arrangement is operable to generate an agent application. Throughout the present disclosure, the term “agent application” as used herein relates to any collection or set of instructions executable by a computer or other digital system so as to configure the computer or the digital system to perform a task that is the intent of the process. Furthermore, the agent application includes one or more routines, data structures, object classes, and/or protocols that support the interaction of an archiving platform and a storage system. It may be appreciated that the agent application may invoke system-level code or calls to other software residing on a server or other location to perform certain functions. Furthermore, the process may be pre-configured and pre-integrated with an operating system, building a software appliance.

Furthermore, the agent application is a software application that operates on any form of computing device, such as the data processing arrangement, and that is capable of accessing static data or resource files associated to the user-viewable hypertext documents on a network, namely the wide area computer network. In an example, the agent application may be a web browser the is operable to retrieve, interpret, render and present web pages from the wide area computer network, commercially available web browser may be Microsoft Internet Explorer®, Google Chrome®, Mozilla Firefox®, and the Opera Browser®. Furthermore, the agent application, namely the web browser may be a computer program and/or routine hosted by the data processing arrangement.

More optionally, the agent application receives the at least one Uniform Resource Identifier (URI). Optionally, the agent application can include one or more sub-routine or set of instruction to acquire the at least one URI. In an example, the sub-routine or set of instruction may generate an input field, namely a location or title bar in the agent application, namely the web browser. In such example, the at least one URI may be entered into the location or title bar via one or more input means by employing text input, voice input, keypad input, and so forth. Furthermore, the one or more input means may include hardware and software components, such as keyboards, mouse, joystick, icons, on-screen keyboards, pull-down menus, buttons, control options and the likes. In such example, the URI may be provided via a virtual keyboard and/or a physical keyboard.

Optionally, the agent application can include an input means to acquire the URI. Optionally, the crawling module receives the at least one URIs from a list of seed URIs. Optionally, the list of seed URIs can be feed to the crawling module manually by an end user. Alternatively, optionally, the list of seed URIs can generate from the history of the web activity of the data processing arrangement.

The crawling module is operable to retrieve source information associated with the at least one Uniform Resource Identifier. The crawling module includes one or more routines to acquire the source information of a user-viewable hypertext document (such as a webpage) associated with the at least one URI. Specifically, the crawling module is operable to acquire the source information included in the agent application that receives the at least one URI and provides the associated user-viewable hypertext document. Throughout the present disclosure, the term “source information” as used herein relates to any program instructions written in a particular programming language, namely source language or a target language. Furthermore, the programming language is typically written in plain text interspersed with formatting instructions. For example, the program instructions may be written using protocol of a particular language such as C®, Java®, Peri®, and PHP®. Furthermore, the program instruction is operable to define features and functioning associated with a webpage. Optionally, the source information may be invoked is operable to call functions and libraries associated thereto.

The source information includes a pool of data elements. Specifically, the source information includes a plurality of data elements that constitute the user-viewable hypertext document. Furthermore, the source information defines the placement and operations of the data element in a user-viewable hypertext document. For example, the user-viewable hypertext document, namely Hypertext Markup Language (HTML, XHTML) document, may include Cascade Style Sheets (CSS), which web page contains content such as text, images, video, audio, etc.

Optionally, the data elements comprise any one of hyperlinks, documents, text, metadata associated with the data elements. Optionally, the data elements comprise a hyperlink, wherein the hyperlink is a feature of a displayed image or text that provides additional information when activated, for example by clicking on the hyperlink. For example, the hyperlink is an image or text that is operable to generate new web content when interacted with. In such an example, the hyperlink may be a URL that points to a different web page contenting additional web content. In an example, the hyperlink is indicated by an HTML HREF attribute. Optionally, the data elements comprise documents to content that structures the user-viewable hypertext document. In an example, in an example, the document may include files, scripts, codes, executable programs, web pages or any other digital data that can be transmitted via a network. Optionally, the data elements comprise text that describes content in the user-viewable hypertext document. For example, the text may describe various attributes of a drug. In such an example, the text may describe a chemical composition of the drug, an organization that manufactures the drug, health problems for which the drug is used for, a method of using the drug, side effects associated with the drug and so forth; it will be appreciated that “drug” here refers to a pharmaceutical preparation that is intended for benevolent medicinal purposes, and not in a context of an illicit narcotics substance. Optionally, the data elements comprise metadata associated with the data elements. The term “metadata” as used herein refers to data which provides information about one or more aspects of a data file (such as the fetched web content). For example, the when was the data element created, accessed, modified, and the likes. The metadata can include a hash of the contents of the data file, as well as additional data relating, for example, to a policy for handling the data file.

The crawling module is operable to determine at least one relevant data element from the pool of data elements. The crawling module includes one or more routines or sets of instructions that are operable to analyse the data elements in the pool of data elements to determine at least one relevant data element. For example, the crawling module may include a software algorithm to analyse the hyperlinks, documents, text, metadata associated with the data elements; optionally, network technical such as Eigenvector analysis are employed, for example as described in a granted European patent EP1700421B1 (Canright et al., Telenor AS).

Furthermore, the determining of the at least one relevant data element includes identifying at least one attribute associated with each data element in the pool of the data elements. The at least one attribute associated with each data element refers to the inherent properties of each of the data element. For example, an attribute of the data element may be that the data elements include the text to be displayed in the user-viewable hypertext document, namely the webpage.

Optionally, the at least one attribute associated with each data element includes a type associate with each data element. Furthermore, a type associated with a data element describes a category to which the data element belongs. For example, a user-viewable hypertext document “X” associated with a URI “Y” may include data element “A”, “B”, “C” and “D”. In such example, the data element “A” may be of a Uniform Resource Locator (URL), data element “B” may be of a Uniform Resource Name (URN), data element “C” may be of an image, data element “D” may be of Cascade Style Sheets (CSS) item. Therefore, the data element “A” and “B” may be links to other user-viewable hypertext document, namely webpage or websites that may be linked to “X”, the data element “C” is of graphics type and the data element “D” is type of data that describe the style of “X”. Optionally, the at least one attribute associated with each data element includes a feature associated with each data element. Furthermore, a feature associated with each data element refers to a characteristic of the corresponding data element. In an example, a feature of the data element “B”, namely a Uniform Resource Locator (URL), may describe the subject matter that “B” relates to, such as pharmaceuticals. In another example, another feature of “B” may be that it includes similar domain name as “X” (wherein “X” is a user-viewable hypertext document associated to a URI “Y”). In yet another, a feature of a data element of “X” may describe a status of the data element.

Furthermore, the determining of the at least one relevant data element includes analyzing the identified at least one attribute, based on predefined qualifier conditions, for detecting a relevance factor for each data element. The analyses of the identified at least one attribute of each of the data elements refers to the technique of evaluating one or more behaviors of the identified at least one attribute. For example, a behavior of an attribute of a data element, such as a hyperlink, may be that the hyperlink provides a connection to a user-viewable hypertext document (namely, a web page). Furthermore, the one or more routine or set of instruction hosted in the crawling module are configured to evaluating one or more behaviors of the identified at least one attribute. For example, the one or more routine or set of instruction may be included in a software program that is configured for evaluating one or more behaviors of the identified at least one attribute. The at least one attribute of each of the data elements are evaluated based on predefined qualifier conditions. Throughout the present disclosure, the term “predefined qualifier conditions” as used herein relates to state and/or circumstance for an element, namely, the at least one attribute, of the system. Furthermore, the predefined qualifier conditions signify the state of the at least one attribute that can be used to qualify a data element associated therein, to be the at least one relevant data element. Optionally, the predefined qualifier conditions for determining of the at least one relevant data element is implemented as one or more sub-routines or set of instruction in the crawling module. In an example, predefined qualifier conditions may be one or more instruction codes of the software program that is configured for evaluating one or more behaviors of the identified at least one attribute.

Optionally, the predefined qualifier conditions include relevant type associate with each data element. Specifically, predefined qualifier conditions describe specific types of the data elements that are to be considered relevant for the system. In an example, the one or more sub-routines or set of instruction in the crawling module may be configured to consider one or more types of the data element, such as a hyperlink, as the relevant type for the system. In an example, the one or more sub-routines or set of instruction in the crawling module may be configured to consider data element having certain extension may be considered as relevant for the system, such as .HTML, .XML and the likes. Optionally, the predefined qualifier conditions includes at least one relevant feature associate with the with each data element. Specifically, the predefined qualifier conditions describe specific features of the data elements that are to be considered relevant for the system. In an example, the one or more sub-routines or set of instruction in the crawling module may be configured to consider one or more features of the data element. In an example, a sub-routine or set of instruction of the crawling module consider feature such as domain name, status as a relevant feature. In an example, the one or more sub-routines or set of instruction in the crawling module may be configured to consider data element having a certain domain name, the status may be considered as relevant for the system. Furthermore, analyzing the identified at least one attribute is used to detect a relevance factor for the each data element. The relevance factor refers to a condition that determines the relation of the data element for the system. Specifically, the relation of the data element for the system can be either relevant or irrelevant. In such instance, the one or more sub-routines or set of instruction in the crawling module uses the predefined qualifier conditions to determine the relevance factor of a specific data element. For example, a data element “V” may be a hyperlink type and may have an HTML status 301 associated therein. In such example, the hyperlink type and the feature HTML status 301 may be considered as predefined qualifier conditions. In such example, the data element “V” may have the relevance factor that is positive, i.e. the data element “V” may be considered relevant for the system.

As mentioned previously, determining the at least one relevant data element includes using the relevance factor to determine the at least one relevant data element from the pool of data elements. The one or more routines and/or the set of instruction included in the crawling module is configured to use the relevance factor to determine the at least one relevant data element from the pool of data elements.

The one or more routines and/or the set of instruction identifies a relevance factor associated with each of the data element the pool of data elements, and thereafter identifies the at least one relevant data element. Additionally, the relevance factor for a given data element is positive or negative, i.e. a data element will be either considered relevant for the system or will be considered non-relevant for the system, wherein relevance is determined relative to a distinguishing threshold value. For example, a URI “K” may be associated with a user-viewable hypertext documents “O” may include a pool of data elements including the data element “I”, “J”, “M” and “N”. In such example, the data element “I” may be a hyperlink type and has a feature of having an HTML status 301 associated therein. In such example, the hyperlink type and the feature HTML status 301 may be considered as predefined qualifier conditions. In such an example, the data element “I” may have the relevance factor that is positive, i.e. the data element “I” may be considered relevant for the system. In such example, the user-viewable hypertext documents “O” may include another data element “J” that is of an image type and has a feature of having an HTML status 400 associated therein. In such example, the image type and the feature HTML status 400 may be considered as non-relevant. In such an example, the data element “J” may have the relevance factor that is negative, i.e. the data element “J” may be considered as not relevant for the system. In such example, the data element “M” may be a hyperlink type and has a feature of having an HTML status 403 associated therein. In such example, the hyperlink type and the feature HTML status 403 may be considered as predefined qualifier conditions. In such example, the data element “M” may have the relevance factor that is negative, i.e. the data element “M” may be considered not relevant for the system. In such example, the data element “N” may be an image type and has a feature of having an HTML status 301 associated therein. In such example, the hyperlink type and the feature HTML status 301 may be considered as predefined qualifier conditions. In such example, the data element “N” may have the relevance factor that is positive, i.e. the data element “N” may be considered relevant for the system.

The crawling module is operable to analyse the at least one relevant data element to determine an importance factor associated therewith. Furthermore, the one or more routines and/or the set of instruction included in the crawling module are configured to identify the importance of each relevant data element of the at least one URI. Optionally, the importance factor assigned to a relevant data element can be a numerical value, i.e. one or more routines and/or the set of instruction assigns a numerical value to each of the relevant data element of the at least one URI. Optionally, the importance factor is determined based on web content associated with the at least one relevant data element. For example, the relevant data elements “I” and “N” may be assigned the numerical values 1 and 2 respectively as importance factors. Furthermore, the web content associated with the at least one relevant data element “I” and “N” can be identified based on the feature associate with the with each data element. In such an example, a feature associated with the data element “I” may describe as link relation to be canonical and a feature associated with the data element “N” may describe as link relation to be rev-canonical. Therefore, the one or more routines and/or the set of instruction may assign the numerical values 1 to the data element “P” and the numerical values 2 to the data element “N”. In such instance, the numerical values 1 is greater than 2, therefore the data element “I” may be more important than “N”.

The crawling module is operable to assign a chronological score to each of the at least one relevant data element based on the determined importance factor thereof. Specifically, the one or more routines and/or the set of instruction included in the crawling module are configured to assign a chronological score to each of the at least one relevant data element based on the determined importance factor. Typically, the chronological score refers to a numerical value that may be used to arrange the at least one relevant data element. In an example, for example to plot a chronological score of a relevant data element may determine its position in a list or a graph. In such example, the relevant data elements “I” and “N” may be assigned the chronological score 1 and 2 respectively. In such example, the chronological score 1 is assigned to the relevant data elements “I” and the chronological score 2 is assigned to the relevant data elements “N” as the data element “f” is more important than “N”.

The crawling module is operable to crawl the each of the at least one relevant data element based on the assigned chronological score thereof. Furthermore, the the one or more routines and/or the set of instruction is configured to crawl the at least one relevant data element based on the assigned chronological score thereof.

In an example, the relevant data elements “I” of the user-viewable hypertext documents “O” associated with the URI “K”, that includes the chronological score 1 may be crawled before the data elements “N” of the user-viewable hypertext documents “O”, that includes the chronological score 2. In such example, the crawling of the relevant data elements “I” and “N” may include collecting the content of multiple files related to the data elements “I” and “N” and thereafter, indexing the content for future use.

According to the present invention, the system comprises a database arrangement that is communicably coupled to the data processing arrangement.

Throughout the present disclosure, the term “database arrangement” as used herein, relates to an organized body of digital information regardless of a manner in which the data or the organized body thereof is represented. Optionally, the database arrangement may be hardware, software, firmware and/or any combination thereof. For example, the organized body of digital information may be in a form of a table, a map, a grid, a packet, a datagram, a file, a document, a list or in any other form. The database arrangement includes any data storage software and systems, such as, for example, a relational database like IBM DB2® and Oracle 9®. Furthermore, the database arrangement includes a software program for creating and managing one or more databases. Optionally, the database arrangement may be operable to support relational operations, regardless of whether it enforces strict adherence to a relational model, as understood by those of ordinary skill in the art. Additionally, the database arrangement is populated by the topic-based web content. Optionally, and the database arrangement is populated by the operational data associated with the URIs and the related information, such as predefined qualifier conditions, at least one relevant data element, and the likes.

The database arrangement is operable to aggregate the at least one relevant data element based on the assigned chronological score. The crawling module is configured to provide the database arrangement with the associated importance factor and chronological score associated with each of the relevant data element. Furthermore, the database arrangement may include programs or sets of instructions that are operable to store the relevant data element based on the chronological score associated therein. In an example, the relevant data elements “I” and “N” may include the chronological score 1 and 2 respectively. In such example, a set of instructions included in the database arrangement may be configured to store the relevant data elements “I” and “N” wherein the relevant data elements “I” is accessed before the relevant data elements “N” while accessing data element chronologically. Optionally, the database arrangement includes a data storage unit, wherein the data storage unit is operable to aggregate the at least one relevant data element based on the assigned chronological score. Throughout the present disclosure, the term “data storage unit” as used herein relates to a physical and/or logical entity that can store data that aggregate the at least one relevant data element based on the assigned chronological score. Optionally, the data storage unit can accumulate the at least one relevant data element in the form of a database, a table, a file, a list, a queue, a heap, a memory, a register, and the likes. Additionally, the data storage unit can reside in one logical and/or physical entity and/or may be distributed between two or more logical and/or physical entities. Optionally, the data storage unit can be periodically updated with the data describing attributes of the crawling process of the URI.

### DETAILED DESCRIPTION OF THE DRAWINGS

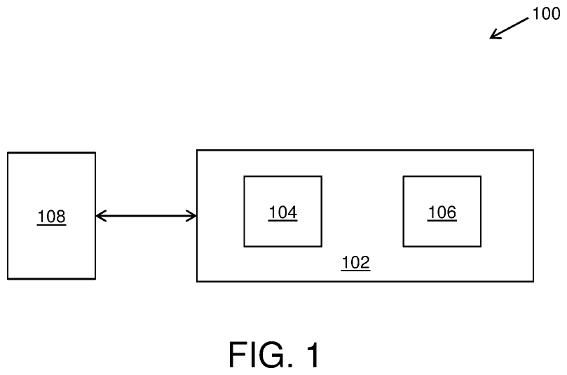

Referring to FIG. 1, there is provided a block diagram illustration of a system 100 that crawls, in accordance with an embodiment of the present disclosure. The system 100 comprises a data processing arrangement 102; optionally, the data processing arrangement 102 includes a combination of custom digital hardware (for example, ASIC's and FPGA's), data processor, data memories, data bus drivers and similar. Furthermore, the data processing arrangement 102 comprises a communication interface 104 and a crawling module 106. Moreover, the communication interface 104 is operable to access a wide area computer network. Furthermore, the crawling module 106 is operable to crawl relevant Unique Resource Identifiers. Additionally, the data processing module 102 is communicably coupled to a database arrangement 108. Furthermore, the database arrangement 108 is operable to aggregate at least one relevant data element based on assigned chronological score.

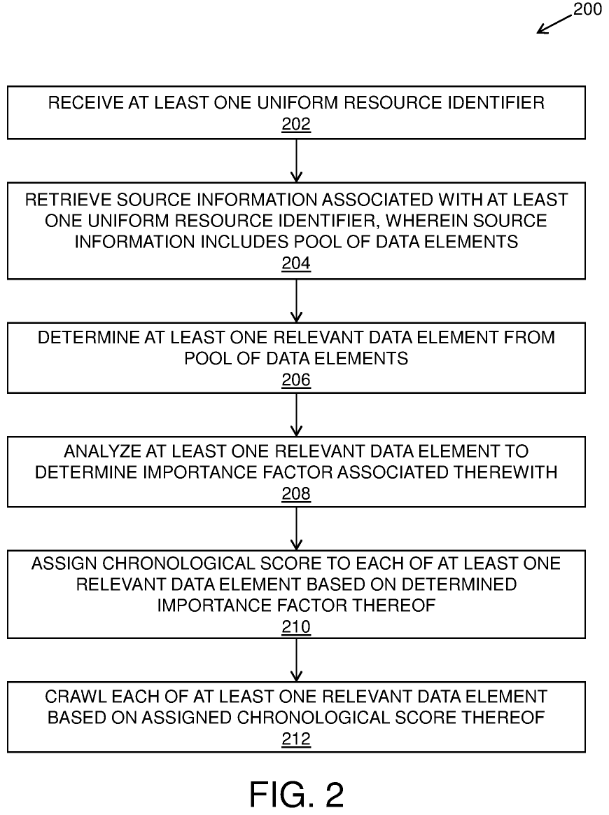

Referring to FIG. 2, there are illustrated therein steps of a method 200 of (for) crawling, in accordance with an embodiment of the present disclosure. At a step 202, at least one Uniform Resource Identifier is received. At a step 204, a source information associated with the at least one Uniform Resource Identifier is retrieved. Furthermore, the source information includes a pool of data elements. At a step 206, at least one relevant data element from the pool of data elements is determined. At a step 208, the at least one relevant data element is analyzed to determine an importance factor associated therewith. At a step 210, a chronological score is assigned to each of the at least one relevant data element based on the determined importance factor thereof. At a step 212, each of the at least one relevant data element is crawled based on the assigned chronological score thereof.

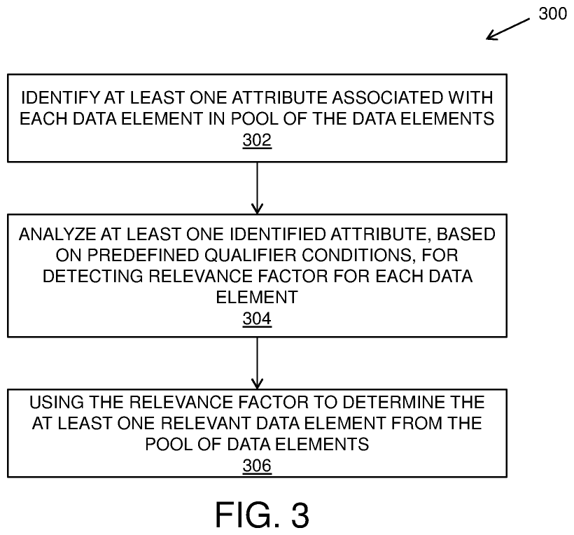

Referring to FIG. 3, illustrated therein are steps of a method 300 of (for) determining the at least one relevant data element, in accordance with an embodiment of the present disclosure. At a step 302, at least one attribute associated with each data element is identified in the pool of the data elements. At a step 304, the at least one identified attribute is analyzed based on predefined qualifier conditions, for detecting a relevance factor for the each data element. At a step 306, the relevance factor is used to determine the at least one relevant data element from the pool of data elements.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system that crawls, wherein the system includes a computer system for executing data processing tasks, wherein the system comprises:
a data processing arrangement comprising a communication interface for accessing a wide area computer network and a crawling module, wherein the crawling module is operable to:
receive at least one Uniform Resource Identifier;
retrieve source information associated with the at least one Uniform Resource Identifier, wherein the source information includes a pool of data elements;
determining at least one relevant data element from the pool of data elements, wherein determining the at least one relevant data element includes:
identifying at least one attribute associated with each data element in the pool of the data elements,
analyzing the at least one identified attribute, based on predefined qualifier conditions, for detecting a relevance factor for the each data element, and
using the relevance factor to determine the at least one relevant data element from the pool of data elements;
analyze the at least one relevant data element to determine an importance factor associated therewith;
assign a chronological score to each of the at least one relevant data element based on the determined importance factor thereof; and
crawl each of the at least one relevant data element based on the assigned chronological score thereof; and
a database arrangement communicably coupled to the data processing arrangement, wherein the database arrangement is operable to aggregate the at least one relevant data element based on the assigned chronological score.

2. The system of claim 1, wherein the crawling module is implemented in a distributed architecture.

3. The system of claim 1, wherein the data processing arrangement is operable to generate an agent application.

4. The system of claim 1, wherein the at least one Uniform Resource Identifier is received at the agent application.

5. The system of claim 1, wherein the data element includes any one of:
hyperlinks, documents, text, metadata associated with the one or more elements.

6. The system of claim 1, wherein the at least one attribute associated with each data element includes any one of:
a type associate with each data element; and
a feature associate with each data element.

7. The system of claim 1, wherein the predefined qualifier conditions is including any one of:
a relevant type associate with each data element; and
at least one relevant feature associate with each data element.

8. The system of claim 1, wherein the importance factor is determined based on web content associated with the at least one relevant data element.

9. The system of claim 1, wherein the database arrangement includes a data storage unit, wherein the data storage unit is operable to aggregate the at least one relevant data element based on the assigned chronological score.

10. A method of (for) crawling, wherein the method includes using a computer system for executing data processing tasks, wherein the method comprises:
(i) receiving at least one Uniform Resource Identifier;
(ii) retrieving source information associated with the at least one Uniform Resource Identifier, wherein the source information includes a pool of data elements;
(iii) determining at least one relevant data element from the pool of data elements, wherein determining the at least one relevant data element includes
identifying at least one attribute associated with each data element in the pool of the data elements,
analyzing the at least one identified attribute, based on predefined qualifier conditions, for detecting a relevance factor for the each data element, and
using the relevance factor to determine the at least one relevant data element from the pool of data elements;
(iv) analyzing the at least one relevant data element to determine an importance factor associated therewith; and
(v) assigning a chronological score to each of the at least one relevant data element based on the determined importance factor thereof; and
(vi) crawling each of the at least one relevant data element based on the assigned chronological score thereof.

11. The method of claim 10, wherein the at least one Uniform Resource Identifier is received at an agent application.

12. The method of claim 10, wherein the data element includes any one of:
hyperlinks, documents, text, metadata associated with the one or more elements.

13. The method of claim 10, wherein the at least one attribute associated with each data element includes any one of:
a type associate with each data element; and
a least one feature associate with each data element.

14. The method of claim 10, wherein the predefined qualifier conditions is including any one of:
a relevant type associate with each data element; and
at least one relevant feature associate with each data element.

15. The method of claim 10, wherein the importance factor is determined based on web content associated with the at least one relevant data element.

16. A computer readable medium, containing program instructions for execution on a computer system, which when executed by a computer, cause the computer to perform method steps of a method of (for) crawling, the method comprising the steps of:
receiving at least one Uniform Resource Identifier;
retrieving source information associated with the at least one Uniform Resource Identifier, wherein the source information includes a pool of data elements;
determining at least one relevant data element from the pool of data elements, wherein determining the at least one relevant data element includes:
identifying at least one attribute associated with each data element in the pool of the data elements,
analyzing the at least one identified attribute, based on predefined qualifier conditions, for detecting a relevance factor for the each data element, and
using the relevance factor to determine the at least one relevant data element from the pool of data elements;
analyzing the at least one relevant data element to determine an importance factor associated therewith;
assigning a chronological score to each of the at least one relevant data element based on the determined importance factor thereof; and
crawling each of the at least one relevant data element based on the assigned chronological score thereof.
